(19) United States
(12) Patent Application Publication
(10) Pub. No.: US 2013/0338538 A1
(43) Pub. Date: Dec. 19, 2013

(54) GUIDE WIRE ARRANGEMENT

(71) Applicant: Agency for Science, Technology and Research, Singapore (SG)

(72) Inventors: Woo Tae PARK, Singapore (SG); Muhammad HAMIDULLAH, Singapore (SG); Ming-Yuan CHENG, Singapore (SG); Cairan HE, Singapore (SG); Li Shiah LIM, Singapore (SG)

(21) Appl. No.: 13/864,220

(22) Filed: Apr. 16, 2013

(30) Foreign Application Priority Data
Apr. 16, 2012 (SG) 201202773-6

(51) Int. Cl. A61B 5/00; A61B 5/06

(52) U.S. Cl. CPC A61B 5/6851; A61B 5/065
USPC 600/585

(57) ABSTRACT

A guide wire arrangement is provided. The guide wire arrangement includes an elongate coil element having two opposing ends; a force transmitting element arranged at one of the two opposing ends of the coil element; a sensor assembly arranged in the coil element at a predefined distance away from the force transmitting element; and a core wire extending from the force transmitting element to the sensor element through the coil element; wherein the coil element is configured to allow the core wire to be moveable relative to the sensor assembly and the sensor assembly is configured to detect movement of the core wire relative to the sensor assembly upon force impact on the force transmitting element.

### CROSS-REFERENCE TO RELATED APPLICATION

This application claims the benefit of priority of Singapore Patent Application No. 201202773-6, filed 16 Apr. 2012, the contents of which being hereby incorporated by reference in its entirety for all purposes.

### TECHNICAL FIELD

Various embodiments relate generally to a guide wire arrangement.

### BACKGROUND

In a catheterization procedure, a guide wire combined with fluoroscopy and low X-ray dose is used by a physician to evaluate or diagnose cardiovascular and endovascular related diseases. Fluoroscopy and low X-ray dose can only be used briefly and intermittently because excessive exposure can increase a person's lifetime risk of developing cancer. Due to that reason, Food and Drug Administration (FDA) announces initiative to reduce radiation exposure from medical imaging. The amount of radiation dose required will depend on the surgeon skill, knowledge, and experience in performing the procedure.

Further, successful passage of the guide wire through the narrowing vascular vessel may depend on the skills and haptic feel of the surgeon. Some cases may be abandoned in case the vascular vessel is being pierced through. Therefore, tactile feedback on a proximal end of a guide wire is desirable.

Tactile feedback on a proximal end of a guide wire can be an important indicator to diagnose abnormalities in blood vessel, such as blood vessels narrowing (stenoses). With quantified and reliable tactile feedback, the surgeon will be less dependent on medical imaging.

Hence, the total exposure time of a person to radiation dose can be reduced. However, the guide wire mechanical tactile feedback is very limited and subjective to the physician performing the procedure. Furthermore, it is highly dependent on its mechanical design where another parameter, steerability for instance, has to be traded off to increase guide wire tactile feedback.

Based on the core wire configuration on the distal end, standard commercial guide wire is mainly divided into two different types shown in FIG. 1: a fixed core guide wire 102 and a movable core guide wire 104. The fixed core guide wire 102 has one single core wire 106 from the proximal end 108 to the distal tip 110 of the guide wire 102 which will enable direct torque transmission from the proximal end 108 to the distal tip 110. The movable core guide wire 104 has a first core wire 112 and a second core wire 114. The first core wire 112 is movable relative to the second core wire 114. The fixed core guide wire 102 may have better mechanical tactile feedback in comparison to the movable core guide wire 104. The movable core guide wire 104 may generally be more flexible and steerable than the fixed core guide wire 102.

### SUMMARY

According to one embodiment, a guide wire arrangement is provided. The guide wire arrangement includes an elongate coil element having two opposing ends; a force transmitting element arranged at one of the two opposing ends of the coil element; a sensor assembly arranged in the coil element at a predefined distance away from the force transmitting element; and a core wire extending from the force transmitting element to the sensor element through the coil element; wherein the coil element is configured to allow the core wire to be moveable relative to the sensor assembly and the sensor assembly is configured to detect movement of the core wire relative to the sensor assembly upon force impact on the force transmitting element.

### BRIEF DESCRIPTION OF THE DRAWINGS

In the drawings, like reference characters generally refer to the same pans throughout The different views. The drawings are not necessarily tc scale, emphasis instead generally being placed upon illustrating the principles of the invention. In the following description, various embodiments of the invention are described with reference to the following drawings, in which:

FIG. 1 shows a schematic diagram of a conventional fixed core guide wire and a conventional movable core guide wire.

FIG. 2 shows a schematic diagram of a guide wire arrangement according to one embodiment.

FIG. 3 shows a schematic diagram of a guide wire arrangement according to one embodiment.

FIG. 4 shows a schematic diagram of a coil element of a guide wire arrangement according to one embodiment.

FIG. 5 shows a schematic diagram of a core wire and a further coil wire of a guide wire arrangement according to one embodiment.

FIG. 6 shows a schematic diagram of a sensor assembly of a guide wire arrangement according to one embodiment.

FIGS. 7A-7C show schematic diagrams of a sensor of a guide wire arrangement according to one embodiment.

FIGS. 8A and 8B show schematic diagrams of a sensor and a core wire of a guide wire arrangement according to one embodiment.

FIGS. 9A-9C show schematic diagrams of a sensor and a core wire of a guide wire arrangement according to one embodiment.

FIG. 10 shows a schematic diagram of a guide wire arrangement according to one embodiment.

FIGS. 11A-11D show a process of forming a sensor assembly of a guide wire arrangement according to one embodiment.

FIGS. 12A-12D show an assembly process of a guide wire arrangement according to one embodiment.

FIG. 13 shows a simulation result of a sensor and a core wire of a guide wire arrangement according to one embodiment.

FIG. 14 shows a simulation result of a sensor and a core wire of a guide wire arrangement according to one embodiment.

### DETAILED DESCRIPTION

Embodiments of a guide wire arrangement will be described in detail below with reference to the accompanying figures. It will be appreciated that the embodiments described below can be modified in various aspects without changing the essence of the invention.

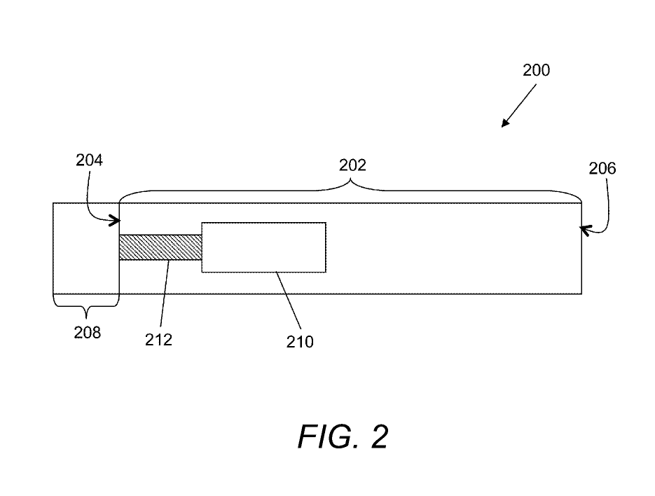

FIG. 2 shows a schematic diagram of a guide wire arrangement 200 according to one embodiment. The guide wire arrangement 200 includes an elongate coil element 202 having two opposing ends 204, 206. The guide wire arrangement 200 includes a force transmitting element 208 arranged at one of the two opposing ends 204, 206 of the coil element 202. The guide wire arrangement 200 includes a sensor assembly 210 arranged in the coil element 202 at a predefined distance away from the force transmitting element 208. The guide wire arrangement 200 includes a core wire 212 extending from the force transmitting element 208 to the sensor assembly 210 through the coil element 202. The coil element 202 is configured to allow the core wire 212 to be moveable relative to the sensor assembly 210. The sensor assembly 210 is configured to detect movement of the core wire 202 relative to the sensor assembly 210 upon force impact on the force transmitting element 208.

In one embodiment, the core wire may extend adjacent to the sensor assembly or through the sensor assembly.

In one embodiment, the guide wire arrangement may include a further core wire extending from another of the two opposing ends of the coil element into the coil element such that a free end portion of the core wire overlaps with a free end portion of the further core wire in a substantially parallel and spaced apart arrangement within the coil element.

In one embodiment, the core wire may be configured to taper in a direction away from the force transmitting element.

In one embodiment, the core wire may be configured to be moveable relative to the further core wire.

In one embodiment, the coil element may include a plurality of coil regions with varying coil wire diameters, spring constants and/or coil spacings.

in one embodiment, the coil element may include first coil region and a second coil region positioned adjacent to the first coil region. The first coil region may be positioned between the force transmitting element and the second coil region and the first coil region may include a coil wire diameter smaller than the second coil region and a coil spacing wider than the second coil region.

In one embodiment, the sensor assembly may include a flexible cable, a sensor, and a chip. The sensor and the chip may be attached onto the flexible cable.

In one embodiment, a portion of the flexible cable upon which the sensor is attached may be arranged between two windings of the coil element with it normal parallel to the longitudinal axis of the coil element.

In one embodiment, the sensor and the chip are attached onto the flexible cable by a flip chip process.

In one embodiment, the sersor may be positioned between the force transmitting element and the chip.

In one embodiment, the sensor assembly may further include a plurality of electrically conductive wires attached to the flexible cable.

In one embodiment, the sensor may include a microelectromechanieal system (MEMS) sensor.

In one embodiment, the sensor may be a ring-shaped sensor including a plurality of suspended beams and a suspended ring connecting the plurality of suspended beams in the centre.

In one embodiment, the core wire may extend from the force transmitting element through the suspended ring of the ring-shaped sensor.

In one embodiment, the plurality of suspended beams may include four suspended beams.

In one embodiment, the sensor may further include a layer of polymer coating disposed over the plurality of suspended beams and the suspended ring.

In one embodiment, the chip may include an application-specific integrated circuit (ASIC).

In one embodiment, the force transmitting element may include a rounded tip structure.

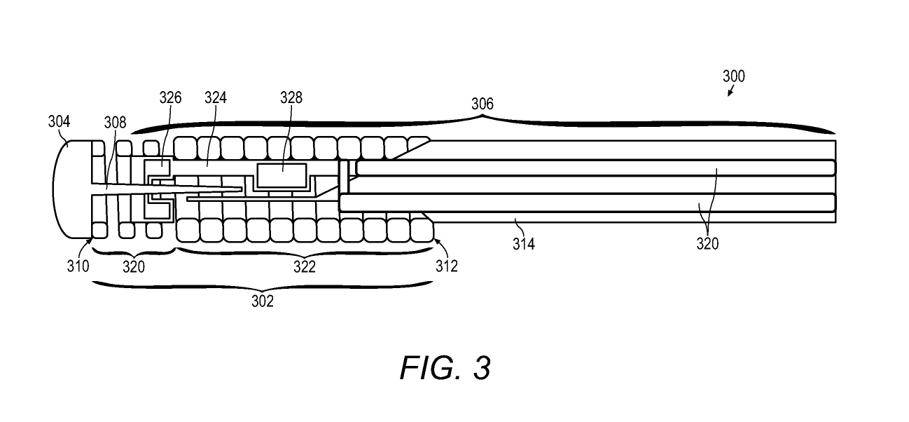

FIG. 3 shows a schematic diagram of a guide wire arrangement 300 according to one embodiment. The guide wire arrangement 300 includes an elongate coil element 302, a force transmitting element 304, a sensor assembly 306 and a core wire 308.

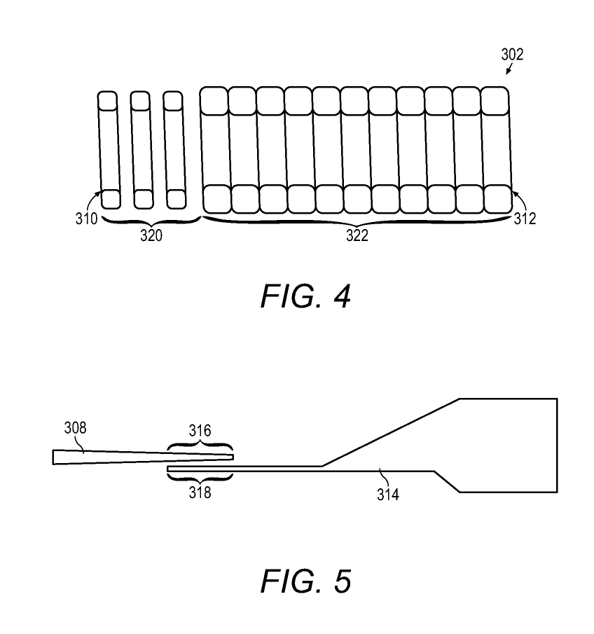

The elongate coil element 302 has two opposing ends (e.g. a first end 310 and a second end 312). The coil element 302 may include a plurality of coil regions with varying coil wire diameters, spring constants and/or coil spacings. In one embodiment, as also shown in FIG. 4, the coil element 302 may have a first coil region 320 and a second coil region 322 positioned adjacent to the first coil region 320. The first coil region 320 may be positioned between the force transmitting element 304 and the second coil region 322. The first coil region 320 may include a coil wire diameter smaller than the second coil region 322 and a coil spacing wider than the second coil region 322. The first coil region 320 of the coil element 302 can be designed to be sparse (e.g. have a wider coil spacing) as compared to the second coil region 322 to provide

In one embodiment, the coil element 302 may have a single coil wire diameter. The coil element 302 may include a spring.

The force transmitting element 304 is disposed at one of the two opposing ends (e.g. the first end 310) of the coil element 302. The force transmitting element 304 may have a rounded tip structure.

In one embodiment, the sensor assembly 306 is arranged in the coil element 302 at a predefined distance away from the force transmitting element 304. The core wire 310 extends from the force transmitting element 304 to the sensor assembly 306 through the coil element 302.

In one embodiment, the core wire 308 may extend adjacent to the sensor assembly 306 or through the sensor assembly 306. The core wire 308 may taper in a direction away from the force transmitting element 304. In other words, a dimension (e.g. diameter) of the core wire 308 may decrease as it extends away from the force transmitting element 304.

In one embodiment, the guide wire arrangement 300 further includes a further core wire 314. The further core wire 314 may extend from another of the two opposing ends (e.g. the second end 312) of the coil element 302 such that a free end portion 316 of the core wire 308 overlaps with a free end portion 318 of the further core wire 314 in a substantially parallel and spaced apart arrangement within the coil element 302. The arrangement of the core wire 308 and the further core wire 314 is more clearly illustrated in FIG. 5. The core wire 308 may be configured to be moveable relative to the further core wire 314.

In one embodiment, the core wire 310 and the further core wire 314 may include steel.

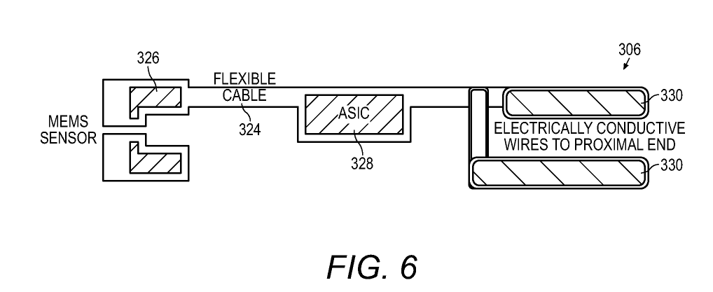

In one embodiment, the sensor assembly 306 includes a flexible cable 324, a sensor 326 and a chip 328. As also shown in FIG. 6, the sensor 326 and the chip 328 are attached onto the flexible cable 324. In one embodiment, the sensor 326 and the chip 323 may be attached onto the flexible cable 324 by a flip chip process. The sensor assembly 306 may also include a plurality of electrically ccnductive wires 330 attached to the flexible cable 324.

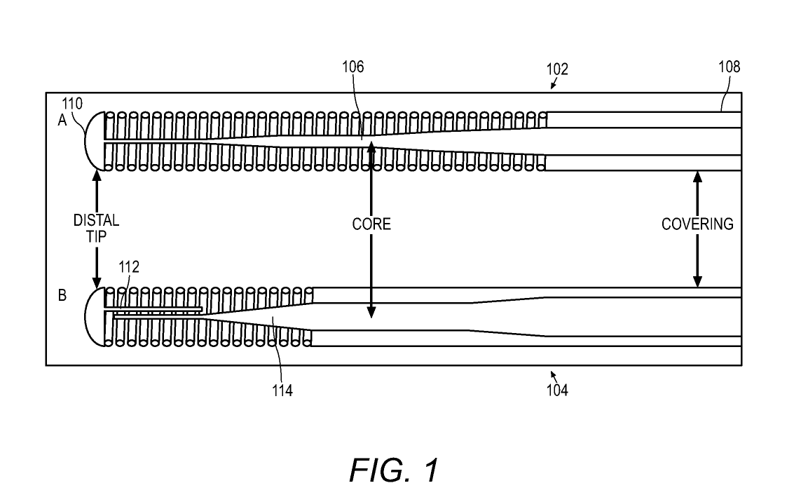

Referring to FIG. 1 a portion of the flexible cable 324 upon which the sensor 326 is attached may be arranged between two windings 332a, 332b of the coil element 302 with its normal 336 parallel to the longitudinal aYis 338 of the coil element 302. The sensor 326 is positioned between the force transmittirg element 304 and the chip 328.

In one embodiment, the sensor 326 is a microelectromechanical system (MEMS) sensor. The sensor 326 may be a MEMS tri-axial force sensor. The chip 328 is an application-specific integrated circuit (AMC).

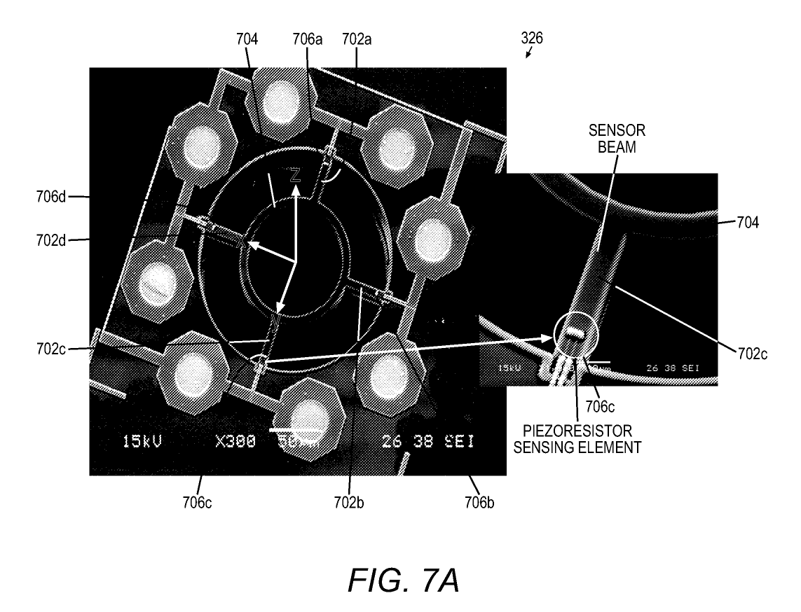

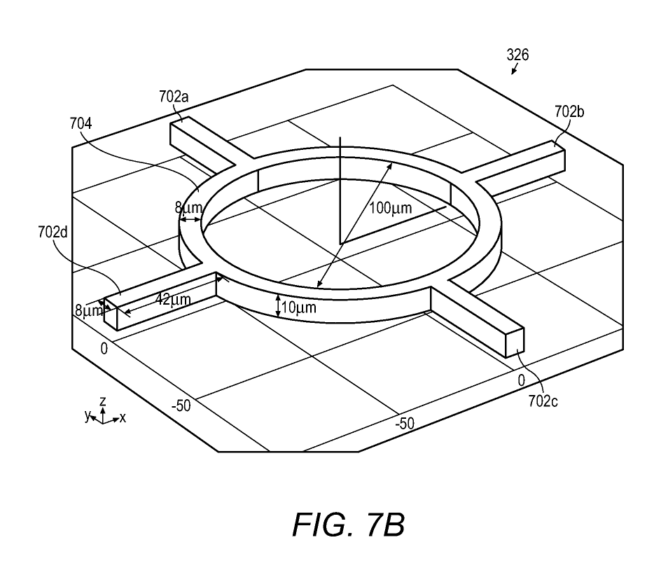

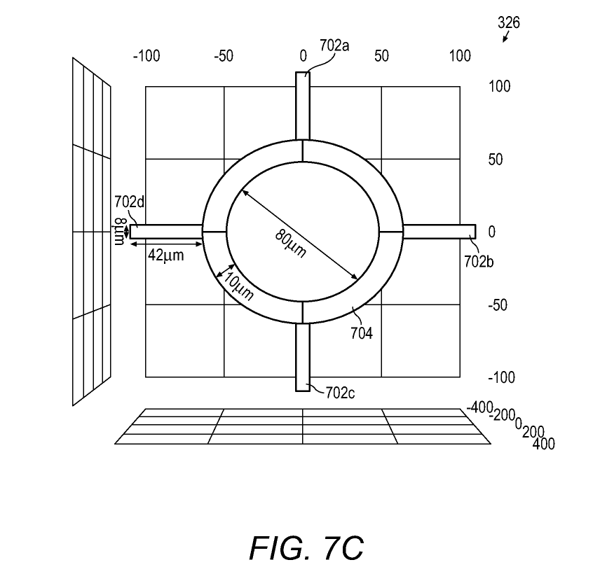

In one embodiment, the sensor 326 is a ring-shaped sensor as shown in FIGS. 7A-7C. The sensor 326 may include a plurality of suspended beams. The plurality of suspended beams may include four suspended beams 702a, 702b, 702c, 702d. The number of suspended beams may vary in different embodiments. The suspended beams 702a, 702b, 702c, 702d may include silicon.

In one embodiment, for guide wire appli-mtion, the size of the sensor 326 may range between about 100 μm to about 1000 μm so that the sensor 326 can be fitted inside a guide wire. Each of the suspended beams 702a, 702b, 702c, 702d may have a length ranging between about 20 μto about 200 μm, a width ranging between about 4 μm to about 40 μm, and a height ranging between about 5 μm to about 50 μm. Each of the suspended beams 702a, 702b, 702c, 702d may have a length of about 42 μm, a width of about 8 μm and a height of about 10 μm.

The sensor 326 may include a suspended ring 704 connecting the suspended beams 702a, 702b, 702c, 702d in the centre. The suspended ring 704 may include silicon. The suspended ring 704 may have a width ranging between about 4 μm to about 40 μm, and a height ranging between about 5 μm to about 50 μm. The suspended ring 704 may have a width of about 8 μm and a height of about 10 μm. The suspended ring 704 may have an internal diameter ranging between about 40 μm to about 400 μm. The suspended ring 704 may have an internal diameter of about 80 μm to about 100 μm.

Further, the sensor 326 may include a respective piezoresistor sensing element 706a, 706b, 706c, 706d placed on the edge of each suspended beam 702a, 702b, 702c, 702d.

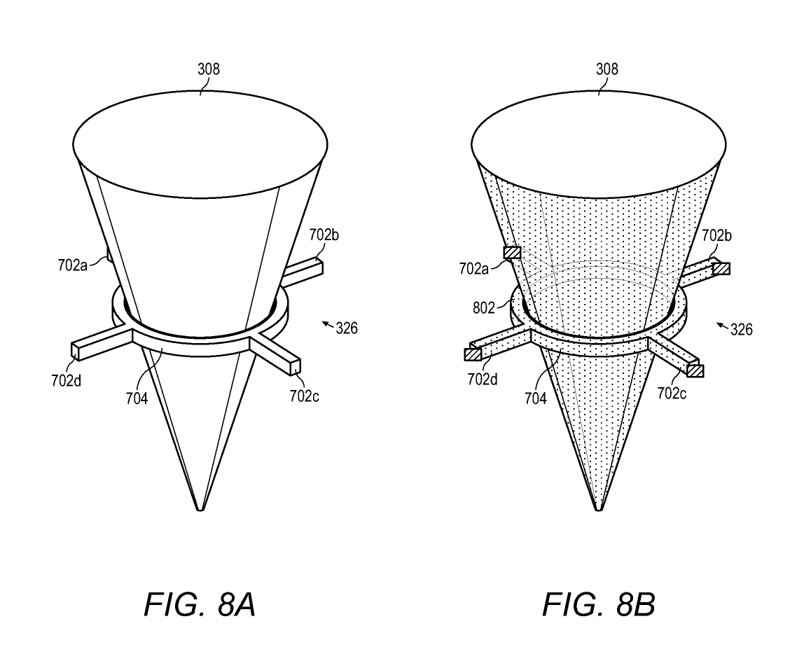

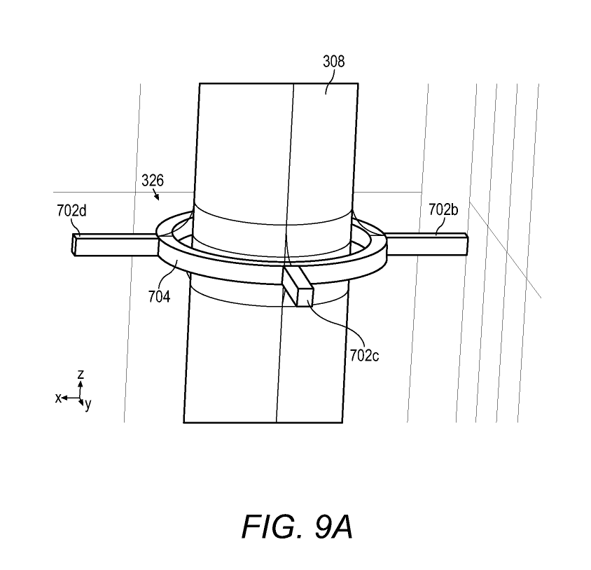

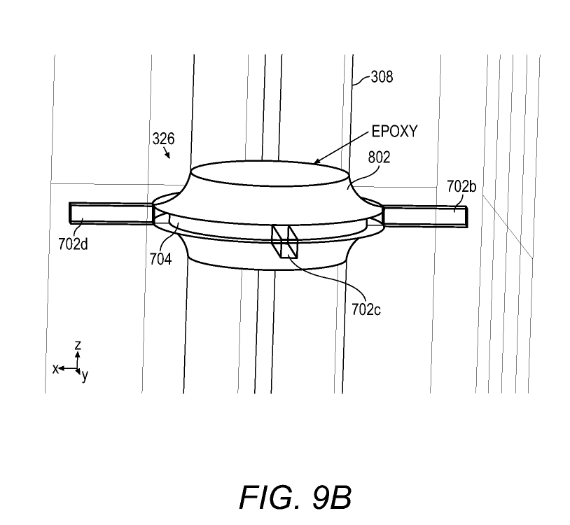

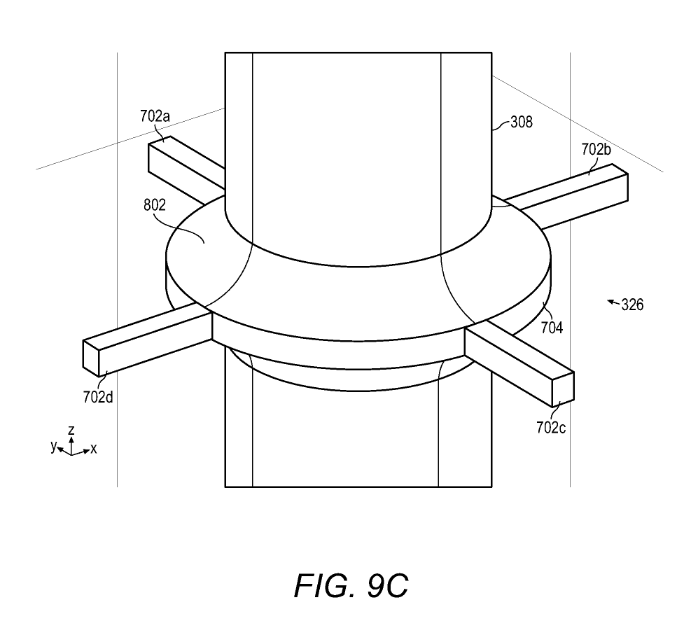

In one embodiment, the core wire 308 may extend from the force transmitting element 304 through the suspended ring 704 of the ring-shaped sensor 326, as shown in FIG. 8A and FIG. 9A. The core wire 308 of FIG. 8A has a tapering diameter. The core wire 308 of FIG. 9A has a constant diameter.

As shown in FIG. 8B, FIG. 9B and FIG. 9C, the sensor 326 may also include a layer of polymer coating 802 disposed over the plurality of suspended beams 702a, 702b, 702c, 702d and the suspended ring 704. The layer of polymer coating 802 may include epoxy. The layer of polymer coating 802 can be used to reduce friction between the plurality of suspended beams 702a, 702b, 702c, 702d and the suspended ring 704 and the core wire 308.

Details of how the guide wire arrangement 300 operates are described in the following.

When a strain occurs on one or more of the suspended beams 702a, 702b, 702e, 702d due to external force, the resistance value of the respective sensing elements 706a, 706b, 706c, 706d will change. For effective force transfer and three dimensional sensing, a rod-like structure is needed in the suspended ring 704 of the sensor 326. For this purpose, a movable tip core guide wire (e.g. including the coil element 302, the force transmitting element 304 and the core wire 308) can be used as a force-transfer element as shown in FIG. 3. rne coil element 302 may be configured to allow the core wire 308 to be moveable relative to the sensor assembly 106, and the sensor assembly 306 may be configured to detect movetrent of the core wire 308 relative to the sensor assembly 306 upon force impact on the force transmitting element 304. The coil element 302 with different spring constants can allow normal and shear force transfer from the force transmitting element 304 to the sensor assembly 306.

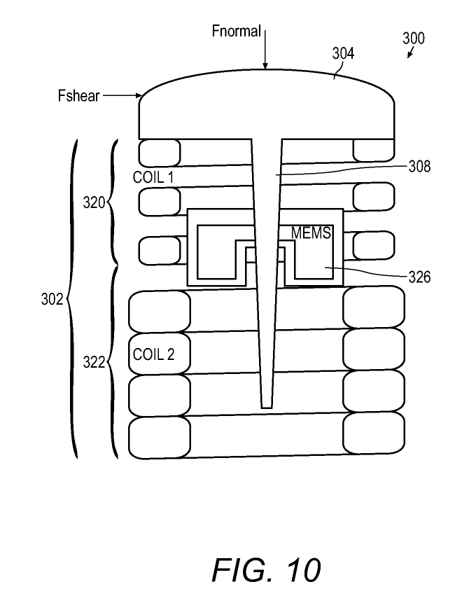

As shown in FIG. 10, normal force (Fₙₒᵣₘₐₗ) and shear force (Fₛₕₑₐᵣ) from a blood vessel during an angioplasty procedure can he transferred from the force transmitting element 304 to the suspended beams 702a, 702b, 702c, 702d. The force may be transferred from the force transmitting element 304 to the suspended beams 702a, 702b, 702c, 702d via a compression of the coil element 302 and the movement of the core wire 308. The suspended beams 702a, 702b, 702c, 702d may be displaced from the respective original positions due to the force transferred from the force transmitting element 304. The normal force (Fₙₒᵣₘₐₗ) and shear force (Fₛₕₑₐᵣ) can be translated into resistance change of the piezoresistor sensing elements 706a, 706b, 706c, 706d. The MEMS sensor signal of the sensor 326 and the ASIC signal of the chip 328 may be integrated and electronically connected by an interconnect metal line (not shown) in the flexible cable 324. The resistance change input signal may be processed by the chip 328 to be sent out to the proximal end (e.g. second end 312) of the guide wire arrangement 300 to be further processed as quantified tactile feedback or display.

The force impact on the force transmitting element 304 can be represented by:

F=−kx

whereby x is the total displacement of the first coil region (d1) and the second coil region (d2), and k is the total spring constant of the first coil region, the sensor (parallel) and the second coil region (series).

The displacement of the suspended beams 702a, 702b, 702c, 702d may be the same as the displacement (d1) of the first coil region 320. The maximum displacement of the suspended beams 702a, 702b, 702c, 702d can be limited by designing the spring constant of the first coil region 320.

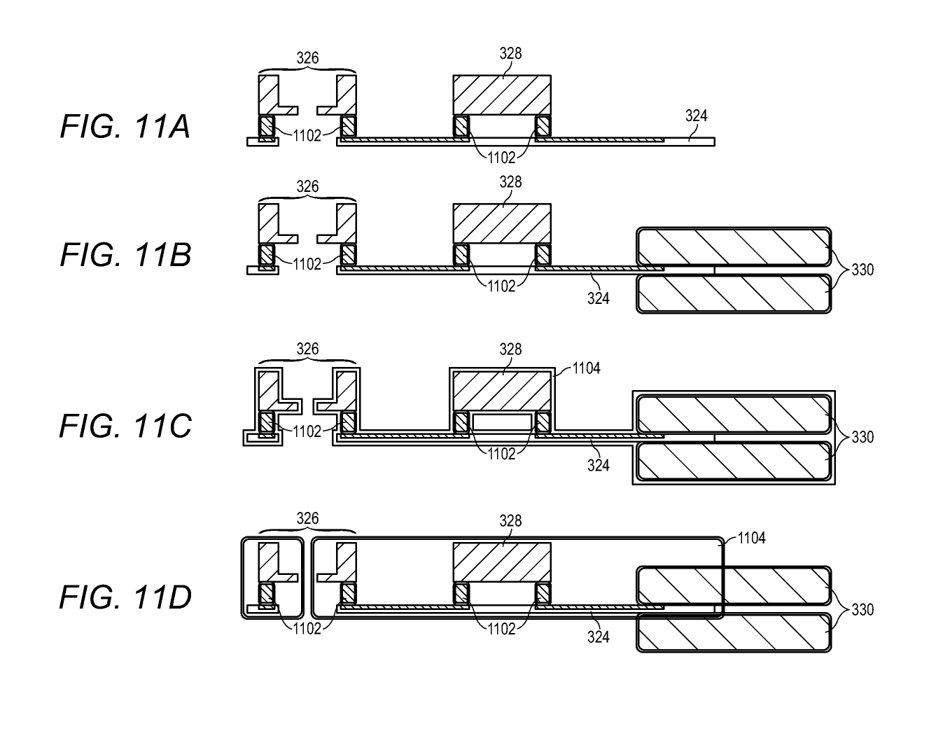

FIG. 11A-D shows a process of forming the sensor assembly 306. FIG. 11A shows that the sensor 326 and the chip 328 are attached onto the flexible cable 324 by a flip chip process. The sensor 326 and the chip 328 may be attached onto the flexible cable 324 via stud bumps 1102. FIG. 11B shows that electrically conductive wires 330 are attached to the flexible cable 324. The wires 330 can send a signal from the ASIC chip 328 to the second end 312 of the guide wire arrangement 300. A polymeric material 1104 may be deposited or coated on the flexible cable 324 to form the sensor assembly 306. The polymeric material 1104 can reduce friction between the silicon material of the sensor 326 (e.g. the ring structure of the sensor 326) and the core wire 310. In one embodiment, the polymeric material 1104 may be deposited or coated by conformal deposition of parylene as shown in FIG. 11C. In one embodiment, the polymeric material 1104 may be deposited or coated by i) encapsulation and laser drilling or ii) precision molding as shown in FIG. 11D.

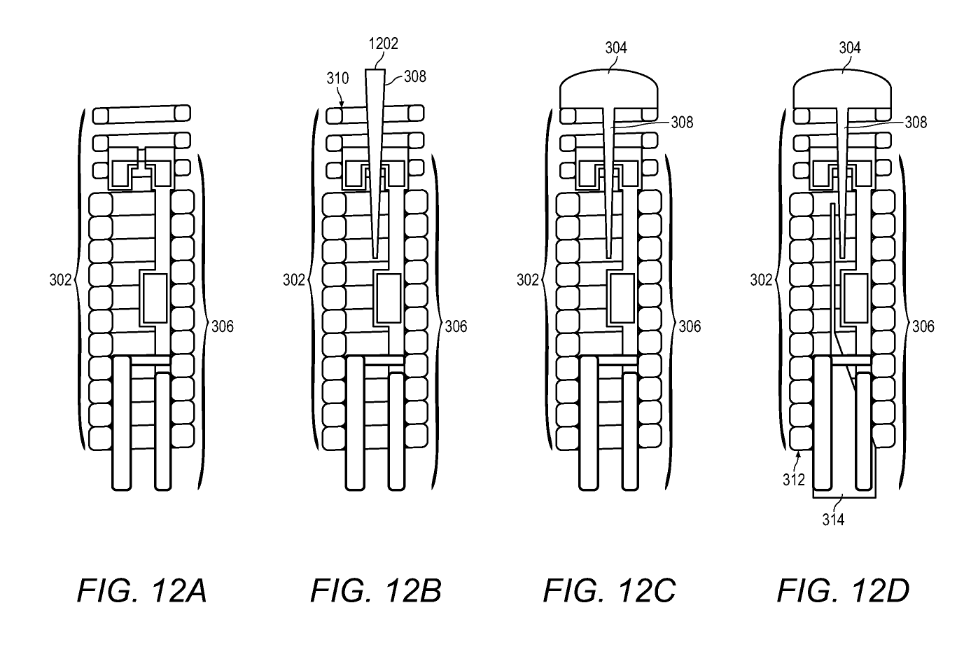

FIG. 12A-D shows an assembly process of the guide wire arrangement 300. FIG. 12A shows that the integrated tactile sensor system (e.g. sensor assembly 306) is placed inside the coil element 302. FIG. 12B shows that the core wire 308 is inserted into the coil element 302. The core wire 308 may be inserted into the coil element 302 from the first end 310 of the coil element. 302. The core wire 308 may be a tapered tip core wire. FIG. 12C shows that one end 1202 of the core wire 308 (see FIG. 12B) is welded to attach the core wire 310 on the coil element 302 and to form a force transmitting element 304. The force transmitting element 304 may have a rounded tip structure. FIG. 12D shows that the further core wire 314 is inserted into the coil element 302 for a complete guide wire arrangement structure. The further core wire 314 may be inserted into the coil element 302 from the second end 312 of the coil element 302.

The assembly process may be compatible with standard commercial guide wire assembly process.

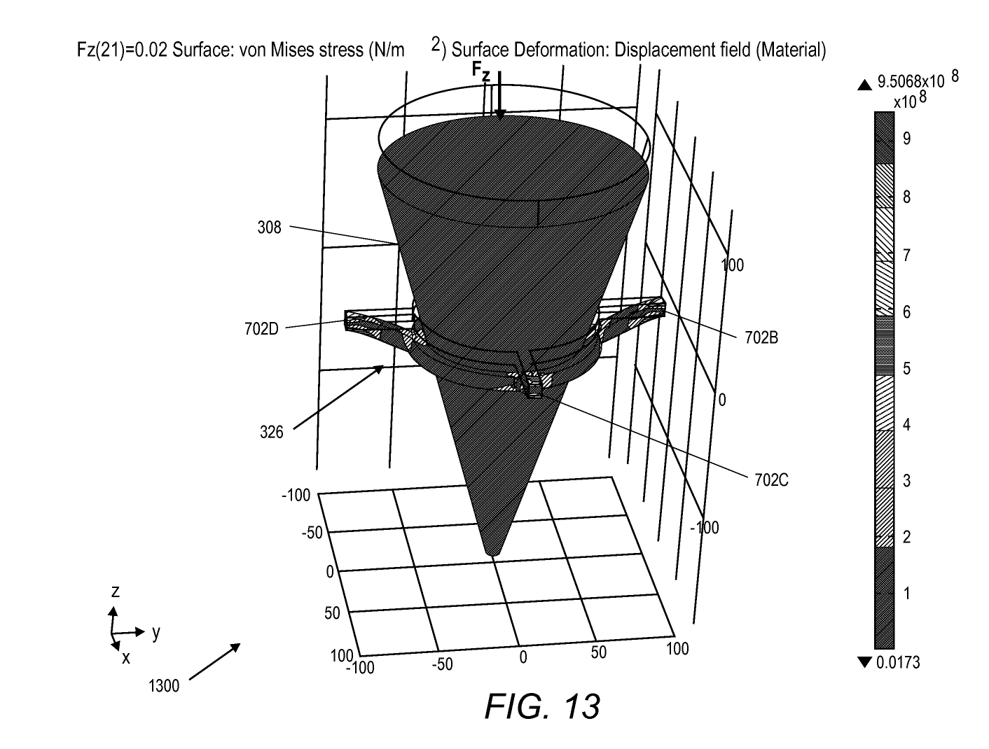

FIG. 13 shows a simulation result 1300 of the sensor 326 and the core wire 310 as illustrated in FIG. 8A. The simulation result 1300 shows the stress distribution on the suspended beams 702a, 702b, 702c, 702d of the sensor 320 and the core wire 310 under a force (F_{z}) of 0.02 N. The simulation result 1300 shows that the maximum stress is located on the edge of the suspended beams 702a, 702b, 702; 702d where the piezoresistor sensing elements 706a, 706b, 706c, 706d are located. The location of the piezoresistor sensing elements 706a, 706b, 706c, 706d on the edge of the suspended beams 702a, 702b, 702c, 702d will optimize the sensitivity of the sensor 326.

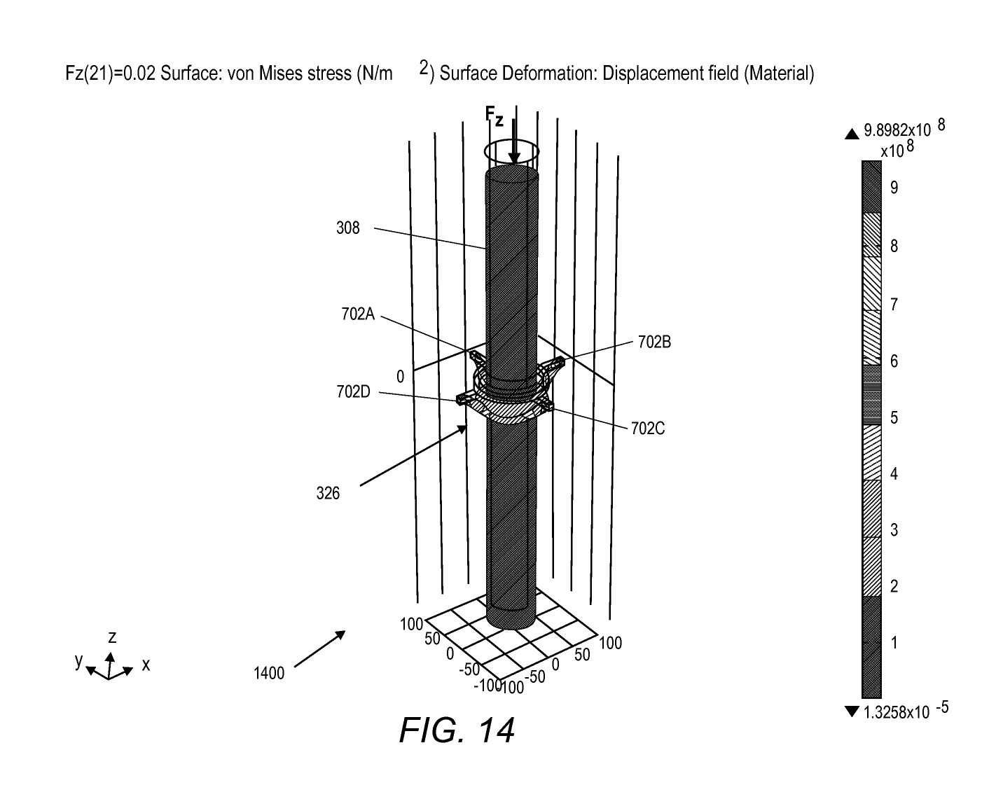

FIG. 14 shows a simulation result 1400 of the sensor 326 and the core wire 310 as illustrated in FIG. 9A. The simulation result 1400 shows the stress distribution on the suspended beams 702a, 702b, 702e, 702d of the sensor 326 and the core wire 310 under a force (F_{z}) of 0.02 N. The simulation result 1400 shows the maximum stress is located on the edge of suspended beams 702a, 702b. 702c, 702d where the piezoresistor sensing elements 706a, 706b, 706c, 706d are located. The location of the piezoresistor sensing elements 706a, 706b, 706c, 706d on the edge of suspended beams 702a, 702b, 702c, 702d will optimize the sensitivity of the sensor 326.

In one embodiment, the guide wire arrangement may include a coil, a core wire and a sensor. The core wire may be fixed to one end of the coil by welding one end of the core wire such that a rounded tip structure is formed at the one end of the coil. The sensor may be arranged within the coil. The sensor may have a ring structure such that another end of the core wire can be inserted through the ring structure of the sensor. The guide wire arrangement may include a further core wire disposed into the coil via another end of the coil. A portion of the further core wire may extend out of the coil for handling purposes during use of the guide wire arrangement. During use of the guide wire arrangement, a force may be exerted on the rounded tip structure when it touches a surface. This force may cause the coil to be compressed and may cause the movement of the core wire. This force may then be transferred to the sensor which may cause a displacement of the sensor. The force transferred to the sensor may be translated into a resistance change signal by the piezoresistive sensing elements of the sensor. The sensor may transmit a signal representing resistance change to a chip. The chip may process the signal received from the sensor and may send out a signal for further processing as quantified tactile feedback or display.

The guide wire arrangement as described above may be a movable core guide wire with integrated tactile sensor system on one end (e.g. distal tip) of the guide wire. The guide wire arrangement as described above may have a movable core sensorised guide wire design. The integrated tactile sensor system may provide electronic tactile feedback to overcome the current tactile feedback limitation in guide wire. The integrated tactile sensor system may include a microelectromechanical system (MEMS) sensor, an application-specific integrated circuit (ASIC) chip and a flexible circuit. The integrated tactile sensor system may be attached on the tip of the movable core guide wire. The MEMS sensor design with e.g. a ring structure, a tapered movable core wire and the coil design can enable force transfer to the MEMS sensor and also, facilitate direct integration of e.g. the MEMS sensor with standard manufacturing process of the guide wire. Electronic tactile feedback/display may be provided on the other end (e.g. proximal end) of the guide wire.

The guide wire arrangement as described above may use a ring shaped tactile sensor to allow core wire insertion into the sensor. Polymer coating on the sensor and the tapered core wire can be used to automatically provide precise alignment and locking feature for assembly and use of the guide wire arrangement.

The guide wire arrangement as described above may have a movable core wire tip for tactile force transfer to the sensor/sensing element. In other words., the rviEMS tactile sensor can be integrated on movable core guide wire to give force/tactile feedback to surgeon.

The sensor system design used in the guide wire arrangement as described above can provide easy integration of existing assembly process. The tapered moving tip core wire used in the guide wire arrangement as described above can provide alignment and alignment feasibility in micro-assembly process.

The guide wire arrangement as described above can to used for all angioplasty procedure.

While embodiments of the invention have been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

What is claimed is:

1. A guide wire arrangement, comprising:
an elongate coil element having two opposing ends;
a force transmitting element arranged at one of the two opposing ends of the coil element;
a sensor assembly arranged in the coil element at a predefined distance away from the force transmitting element; and
a core wire extending from the force transmitting element to the sensor assembly through the coil element;
wherein the coil element is configured to allow the core wire to be moveable relative to the sensor assembly and the sensor assembly is configured to detect movement of the core wire relative to the sensor assembly upon force impact on the force transmitting element.

2. The guide wire arrangement of claim 1, wherein the core wire extends adjacent to the sensor assembly or through the sensor assembly.

3. The guide wire arrangement of claim 1, further comprising a further core wire extending from another of the two opposing ends of the coil element into the coil element such that a free end portion of the core wire overlaps with a free end portion of the further core wire in a substantially parallel and spaced apart arrangement within the coil element.

4. The guide wire arrangement of claim 1, wherein the core wire is configured to taper in a direction away from the force transmitting element.

5. The guide wire arrangement of claim 3, wherein the core wire is configured to be moveable relative to the further core wire.

6. The guide wire arrangement of claim 1, wherein the coil element comprises a plurality of coil regions with varying coil wire diameters, spring constants and/or coil spacings.

7. The guide wire arrangement of claim 1, wherein the coil element comprises a first coil region and a second coil region positioned adjacent to the first coil region, wherein the first coil region is positioned between the force transmitting element and the second coil region and the first coil region comprises a coil wire diameter smaller than the second coil region and a coil spacing wider than the second coil region.

8. The guide wire arrangement of claim 1, wherein the sensor assembly comprises :
a flexible cable;
a sensor; and
a chip;
wherein the sensor and the chip are attached onto the flexible cable.

9. The guide wire arrangement of claim 8, wherein a portion of the flexible cable upon which the sensor is attached is arranged between two windings of the coil element with its normal parallel to the longitudinal axis of the coil element.

10. The guide wire arrangement of claim 8, wherein the sensor and the chip are attached onto the flexible cable by a flip chip process.

11. The guide wire arrangement of claim 8, wherein the sensor is positioned between the force transmitting element and the chip.

12. The guide wire arrangement of claim 8, wherein the sensor assembly further comprises a plurality of electrically conductive wires attached to the flexible cable.

13. The guide wire arrangement of claim 8, wherein the sensor comprises a microelectromechanical system (MEMS) sensor.

14. The guide wire arrangement of claim 8, wherein the sensor is a ring-shaped sensor comprising:
a plurality of suspended beams; and
a suspended ring connecting the plurality of suspended beams in the centre.

15. The guide wire arrangement of claim 14, wherein the core wire extends from the force transmitting element through the suspended ring of the ring-shaped sensor.

16. The guide wire arrangement of claim 14, wherein the plurality of suspended beams comprises four suspended beams.

17. The guide wire arrangement of claim 14, wherein the sensor further comprises a layer of polymer coating disposed over the plurality of suspended beams and the suspended ring.

18. The guide wire arrangement of claim 8, wherein the chip comprises an application-specific integrated circuit (ASIC).

19. The guide wire arrangement of claim 1, wherein the force transmitting element comprises a rounded tip structure.
